(19) United States
(12) Patent Application Publication
(10) Pub. No.: US 2021/0045707 A1
(43) Pub. Date: Feb. 18, 2021

(54) APPARATUS AND METHOD FOR ADJUSTING EXPOSURE FOR AN X-RAY DEVICE

(71) Applicant: SOCIEDAD ESPANOLA DE ELECTROMEDICINA Y CALIDAD, S.A., Madrid (ES)

(72) Inventors: Konstantin Sosenko, San Sebastian de los Reyes (ES); Manuel Desco Menéndez, Madrid (ES); Juan Manuel Arco Casanova, Madrid (ES); Mónica Abella García, Madrid (ES); José Maria Ortega, Madrid (ES)

(21) Appl. No.: 16/994,620

(22) Filed: Aug. 16, 2020

Related U.S. Application Data
(60) Provisional application No. 62/887,868, filed on Aug. 16, 2019.

(51) Int. Cl. A61B 6/00; A61B 6/06

(52) U.S. Cl. CPC A61B 6/542; A61B 6/488; A61B 6/06; A61B 6/5282

(57) ABSTRACT

An x-ray imaging system is provided, the system comprising an x-ray source configured to emit an x-ray beam towards an x-ray imaging detector. The x-ray imaging detector is configured to obtain an x-ray image of an object placed adjacent to the x-ray imaging detector and at least partially within a path of the x-ray beam. The system further includes a pre-exposure device positioned between said object and the x-ray source. The pre-exposure device comprises a blocking component having an arrangement of openings across an extent of the component. The pre-exposure device has an active state wherein said blocking component is configured to reside within the path of the x-ray beam, and an inactive state wherein said blocking component is configured to not reside in the path of the x-ray beam.

### CROSS-REFERENCE TO RELATED APPLICATIONS

This application claims priority to U.S. Provisional Patent Application No. 62/887,868, filed on Aug. 16, 2019, the contents of the application is fully incorporated herein by reference.

### TECHNICAL FIELD

This application generally relates to x-ray devices and more specifically, to adjusting exposure for x-ray devices.

### BACKGROUND

X-ray diagnostic devices obtain an image of the internal organs of a patient by emitting an x-ray beam towards the patient and using a detector positioned behind the patient to detect or register the portion(s) of the x-ray beam that penetrate through the body of the patient. The quality of the x-ray image, among other factors, depends on the amount of ionizing radiation that reaches the detector. If the radiation amount is too low or too high, the image quality is lower. In such cases, an additional exposure may be required to increase the image quality, which, in turn, increases the radiation load on the patient. Thus, selecting an appropriate intensity level for the x-ray beam is an important consideration when performing medical x-ray imaging.

There are various existing methods for choosing the appropriate intensity level for a given x-ray beam and other exposure factors. One method is to use a pre-defined table of exposure factors, which determines the correct exposure factor depending on the organ (and its projection) to be imaged and the approximate size of the patient. However, this method is not sensitive to the personal properties of the patient, such as, for example, differences in the x-ray transparency of various bodies due to the specific muscle and fat composition in each body.

Another method for choosing the right exposure factors involves using a dosimetry device (i.e. Automatic Exposure Control (AEC) chamber) located between the patient and the detector. This dosimetry device measures the quantity of radiation which has passed through the patient and reached the detector during exposure. When a threshold amount of radiation is reached, the dosimetry device sends a signal to the x-ray apparatus to terminate the exposure, thus providing an optimal x-ray dose depending on the body density. One disadvantage of this method, however, is that the dosimetry device has fixed spatial position and size, thus requiring accurate positioning of the patient relative to the dosimetry device. Accordingly, any mistake in positioning can lead to incorrect exposure, which may result in a non-diagnostic image.

Another factor that influences the quality of an x-ray image is scattered radiation. Due to the nature of x-rays, there are three types of x-ray interaction with the body. The first type is absorption, or when the energy of the x-ray photon is fully absorbed by the body, or structures within the body, and does not impact the detector. The second type is no interaction, or when the x-ray photon impacts the x-ray detector behind the body without first interacting with the body. The third type of interaction is scatter, or when the x-ray photon energy is partially absorbed by the body structures and the trajectory of the photon is changed. The first and second types of interaction are useful in the imaging procedure because the proportion between photons that pass-through (i.e. produced by the second type of interaction) and photons that are absorbed (i.e. produced by the first type of interaction) represents the density of the body structures, and this body density is measured during the radiology imaging procedures. The third type of interaction, however, does not add information but rather, reduces an overall contrast of the x-ray image because scatter has a chaotic or unpredictable character.

Typically, anti-scatter grids are positioned between the patient and the x-ray detector to minimize the impact of scatter on the image contrast. The anti-scatter grid positioned between the patient and the x-ray detector is intended to absorb x-ray photons with a trajectory that is other than directly from a focal spot of the x-ray source (i.e. produced by the third type of x-ray interaction). One disadvantage of the anti-scatter grid is that the grid also partially absorbs useful x-ray photons (i.e. from the second type of interaction) and because of that, the overall x-ray dose provided to the patient must be increased. Another disadvantage is that the anti-scatter grid must be positioned perpendicular to a central axis of the x-ray source, or the line that runs from a center of the x-ray source to a center of the front surface of the anti-scatter grid. If aligned differently, the amount of photons absorbed by the anti-scatter grid (i.e. from the second type of interaction) will be greater than intended or desired, thus reducing the image quality. Yet another disadvantage of the anti-scatter grid is that the distance between the x-ray source and the anti-scatter grid must be maintained as specified by the grid manufacturer. If that is not the case, the amount of photons absorbed by the anti-scatter grid will likely be greater than what is intended or desired, thus reducing the image quality.

Accordingly, there is still a need in the art for improved x-ray imaging techniques for determining the correct exposure factors for a given patient, or imaging scenario, and producing a high-quality x-ray image, while also reducing the total amount of x-ray exposure.

### SUMMARY

The invention is intended to solve the above-noted and other problems by providing apparatus, system, and method configured to minimize the impact of scattered radiation and optimize exposure parameters depending on a body density of the patient during radiology procedures. In particular, embodiments include placing a pre-exposure device, or “beam stopper,” between an output end of the x-ray source and the patient, and within the path of the x-ray beam, during a “pre-pulse,” or the acquisition of a preliminary exposure image, and removing the pre-exposure device from the x-ray beam before a full pulse, or the acquisition of a main exposure image. The pre-exposure device comprises an x-ray blocking material with x-ray transparent areas, preferably openings or voids of the x-ray blocking material, arranged across the blocking material to allow partial passage of the x-ray beam through the pre-exposure device, thus reducing the amount of exposure to the patient during the pre-pulse. The pre-exposure image may be used to optimize the exposure parameters to the individual body density of the patient and calculate a scatter image representing the scattered radiation produced by the pre-pulse. The main exposure may be performed using the optimized exposure parameters, thus exposing the patient to only a minimally required amount of radiation. In addition, the scatter image may be used to remove scattered radiation from the main exposure image, thus improving the image contrast. Moreover, the pre-exposure image may be used to confirm proper positioning of the x-ray imaging detector relative to the patient, or object to be imaged. If the detector is not properly aligned, the main exposure may be automatically canceled, so that the operator can re-align the object and/or detector.

For example, one embodiment provides an x-ray imaging system comprising an x-ray source configured to emit an x-ray beam towards an x-ray imaging detector, the x-ray imaging detector configured to obtain an x-ray image of an object placed adjacent to the x-ray imaging detector and at least partially within a path of the x-ray beam. The system further comprises a pre-exposure device positioned between the object and the x-ray source and comprising a blocking component that includes an arrangement of openings across an extent of the component. The pre-exposure device has an active state wherein the blocking component is configured to reside within the path of the x-ray beam and an inactive state wherein the blocking component is configured to not reside in the path of the x-ray beam.

Another example embodiment provides an x-ray apparatus comprising an x-ray source configured to emit an x-ray beam, an x-ray imaging detector configured to obtain an x-ray image of an object placed adjacent to the x-ray imaging detector and at least partially within a path of the x-ray beam, a collimator disposed adjacent to an output end of the x-ray source, and a pre-exposure device positioned between said object and the x-ray source and comprising a blocking component including an arrangement of a plurality of openings across an extent of the component. The blocking component is selectively movable to a first position for placing the component within the path of the x-ray beam and to a second position for removing the component from the path of the x-ray beam.

Another example embodiment provides a method of adjusting exposure in an x-ray imaging system comprising at least one controller, an x-ray imaging detector, an x-ray source configured to emit an x-ray beam towards the x-ray imaging detector, and a pre-exposure device positioned adjacent the x-ray source and within a path of the x-ray beam, wherein the pre-exposure device includes a blocking component having an arrangement of openings across an extent of the component to partially block the x-ray beam. The method comprises activating the pre-exposure device using the at least one controller; acquiring a pre-exposure image, using the x-ray imaging detector, while the pre-exposure device is active; deactivating the pre-exposure device using the at least one controller; and acquiring a main exposure image, using the x-ray imaging detector, while the pre-exposure device is inactive.

As will be appreciated, this disclosure is defined by the appended claims. The description summarizes aspects of the embodiments and should not be used to limit the claims. Other implementations are contemplated in accordance with the techniques described herein, as will be apparent to one having ordinary skill in the art upon examination of the following drawings and detail description, and such implementations are intended to within the scope of this application.

### BRIEF DESCRIPTION OF THE DRAWINGS

For a better understanding of the invention, reference may be made to embodiments shown in the drawings identified below. The components in the drawings are not necessarily to scale and related elements may be omitted, or in some instances proportions may have been exaggerated, so as to emphasize and clearly illustrate the novel features described herein. In addition, system components can be variously arranged, as known in the art. Further, in the drawings, like reference numerals designate corresponding parts throughout the several views.

FIG. 1A is a schematic diagram of an exemplary x-ray apparatus comprising a pre-exposure device, in accordance with certain embodiments.

FIGS. 1B and 1C are schematic diagrams of the exemplary x-ray apparatus of FIG. 1A showing the pre-exposure device in alternative positions, in accordance with certain embodiments.

FIG. 2 is a schematic diagram of an exemplary blocking component of a pre-exposure device, in accordance with certain embodiments.

FIG. 3A is a schematic diagram of an exemplary pre-exposure device in an active state, in accordance with certain embodiments.

FIG. 3B is a schematic diagram of the pre-exposure device of FIG. 3A in an inactive state, in accordance with certain embodiments.

FIG. 4 is a flow diagram of an example method for adjusting exposure in an x-ray imaging system, in accordance with certain embodiments.

FIG. 5 is a flow diagram of an example method for analyzing a position of an x-ray detector in an x-ray imaging system, in accordance with certain embodiments.

FIG. 6 is a block diagram of an example x-ray imaging system, in accordance with certain embodiments.

FIG. 7 is an exemplary scatter radiation map acquired by using the x-ray apparatus of FIG. 1A while the pre-exposure device is active, in accordance with certain embodiments.

FIG. 8 is an exemplary scatter image estimated from the scatter radiation map of FIG. 7 in accordance with certain embodiments.

FIG. 9 is an exemplary main exposure image acquired by using the x-ray apparatus of FIG. 1A while the pre-exposure device is inactive, in accordance with certain embodiments.

FIG. 10 is an exemplary corrected x-ray image obtained by subtracting the scatter image shown in FIG. 8 from the main exposure image shown in FIG. 9, in accordance with certain embodiments.

FIG. 11A is a front view of another exemplary blocking component, in accordance with certain embodiments.

FIG. 11B is a close-up view of the blocking component shown in FIG. 11A, in accordance with certain embodiments.

FIG. 12 is a side view of the blocking component shown in FIG. 11A, in accordance with certain embodiments.

### DETAILED DESCRIPTION OF EXAMPLE EMBODIMENTS

While the invention may be embodied in various forms, there are shown in the drawings, and will hereinafter be described, some exemplary and non-limiting embodiments, with the understanding that the present disclosure is to be considered an exemplification of the invention and is not intended to limit the invention to the specific embodiments illustrated.

In this application, the use of the disjunctive is intended to include the conjunctive. The use of definite or indefinite articles is not intended to indicate cardinality. In particular, a reference to “the” object or “a” and “an” object is intended to denote also one of a possible plurality of such objects.

In the following description, elements, circuits and functions may be shown in block diagram form in order to not obscure the present disclosure in unnecessary detail. Additionally, block definitions and partitioning of logic between various blocks is exemplary of a specific embodiment. Further, those of ordinary skill in the art will understand that information and signals as depicted in the block diagrams may be represented using any variety of different technologies or techniques. For example, data, instructions, signals or commends may be represented in the figures, and which also would be understood as representing voltages, currents, electromagnetic waves or magnetic or optical fields, or combinations thereof. Additionally, some drawings may represent signals as a single signal for clarity of the description; and persons skilled in the art would recognize that the signal may represent a bus of signals. Various illustrative logic blocks, modules and circuits described in connection with embodiments disclosed herein may be implemented or performed with one or more processors. As would be appreciated and understood by persons of ordinary skill in the art, disclosure of separate processors in block diagrams may indicate a plurality of processors performing the functions or logic sequence disclosed herein, or may represent multiple functions or sequence performed on a single processor.

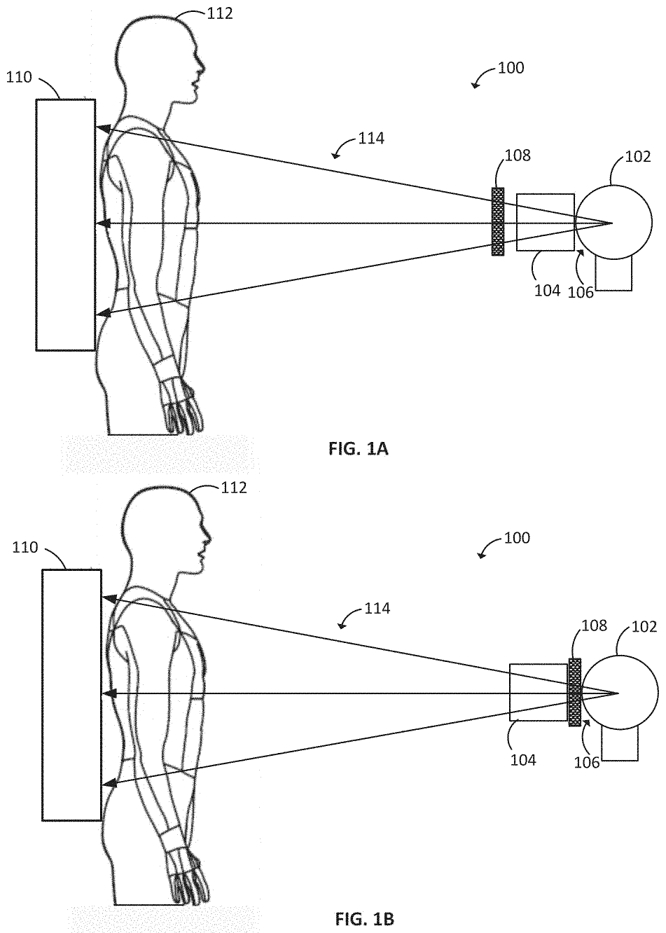

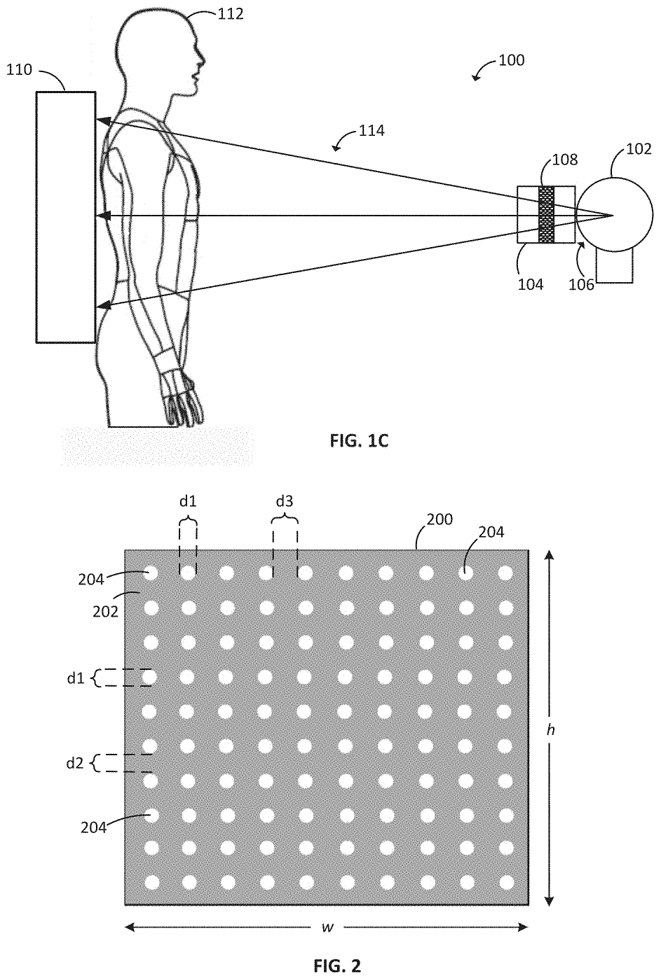

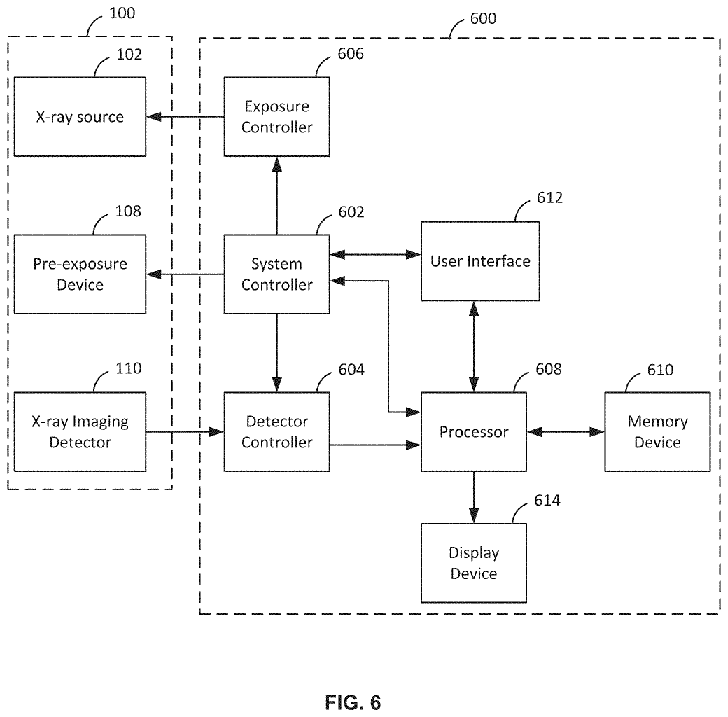

FIG. 1A illustrates an exemplary x-ray apparatus 100 comprising an x-ray source 102 (also referred to herein as “x-ray tube”), a collimator 104 coupled, or disposed adjacent, to an output end 106 (or emitting portion) of the x-ray tube 102, and a pre-exposure device 108 disposed adjacent to, or within, the collimator 104, in accordance with embodiments. Though not shown, the x-ray apparatus 100 may also include an x-ray generator for providing high voltage power to the x-ray tube 102 and one or more filters for removing any unnecessary or unusable parts of the x-ray output produced by the x-ray source 102. The x-ray apparatus 100 may also include, or be coupled to, a control unit or operating console for allowing user control of x-ray tube current (mA) and voltage (kV) parameters, as well as exposure time (mS), and at least one controller configured to adjust one or more exposure control parameters based on said user control inputs and/or other information (e.g., as shown in FIG. 6).

As shown in FIG. 1A, the x-ray apparatus 100 further includes an x-ray imaging detector 110 disposed a pre-determined distance from the x-ray tube 102. The detector 110 may be placed behind an object 112 to be imaged (e.g., a human patient), or on an opposite side of the object 112 than the x-ray tube 102. For example, the object 112 may be arranged on, against, or adjacent to a front face of the detector 110, between the detector 110 and the pre-exposure device 108. The distance between the detector 110 and the x-ray tube 102 may be pre-defined by an image acquisition protocol being used for, or applied to, a given application, as will be appreciated. The detector 110 may be a flat panel detector or any other suitable x-ray imaging detector.

During operation, the x-ray source 102 can be configured to emit an x-ray beam 114 towards the object 112, and the detector 110 disposed behind it. The x-ray imaging detector 110 can be configured to obtain an x-ray image of the object 112, or more specifically, the portion or region of the object 112 that coincides with a path of the x-ray beam 114. For example, as shown in FIG. 1A, the object 112 may be arranged relative to the detector 110 so that only the portion of the object 112 that will be imaged (e.g., a particular organ or region of the body) is within the path of the x-ray beam 114.

As will be appreciated, the x-ray tube 102 generates the x-ray beam, or x-radiation, by converting electron energy into photons. More specifically, the x-ray tube 102 includes a cathode and an anode. As electrical current flows through the tube 102 from the cathode to the anode, the electrons undergo an energy loss, which results in the generation of x-radiation. The quantity (or exposure) and quality (or spectrum) of the resulting x-radiation can be controlled by adjusting certain parameters that control the x-ray production process (also referred to herein as “exposure control parameters”). These include the voltage or electrical potential (measured in kilo-Volts (kV)) that is applied to the x-ray tube 102, the electrical current (measured in milli-Amps (mA)) that flows through the x-ray tube 102, and the exposure time or duration (measured in milli-seconds (mS)) of the x-ray tube 102. The electrical potential (kV) determines the amount of energy carried by each electron emitted from the cathode, and the electrical current (also referred to herein as “anode current”) determines the number or quantity of electrons that strike the anode.

Some x-ray tubes also allow the operator to select a focal spot size, or a size of the area on the surface of the anode where x-ray radiation is produced. The exact dimensions of the focal spot are determined by the dimensions of the electron beam arriving from the cathode and can vary depending on the design of the particular x-ray tube. Typically, the focal spot is approximately rectangular with dimensions ranging from 0.1 to 2 millimeter (mm). As will be appreciated, small focal spots produce less blurring and high visibility of details in the x-ray image, while large focal spots have a greater heat-dissipating capacity. Most x-ray tubes have two focal spot sizes (e.g., large and small), and the operator can select the appropriate focal spot size for a given application. For example, a small focal spot may be selected when the object to be imaged is small and thin and therefore, requires a relatively low amount of radiation, and/or when high image visibility of details is essential.

The collimator 104 is configured to minimize the field of radiation by narrowing the x-ray beam 114 to a select size as the beam 114 exits the x-ray tube 102. As an example, the collimator 104 may be comprised of a series of metal leaves or blades (e.g., tungsten) that can overlap to create different-sized openings, or fields. The opening of the collimator 104 can be automatically, or manually, adjusted based on a size of the detector 110. The size and/or shape of the collimator opening can be selected so that the portion of the x-ray beam 114 that reaches the detector 110 generally coincides in size with that of the detector 110, for example, as shown in FIG. 1A. That is, the collimator 104 can be configured to narrow the x-ray beam 114 to a size that is selected based on the size of the detector 110.

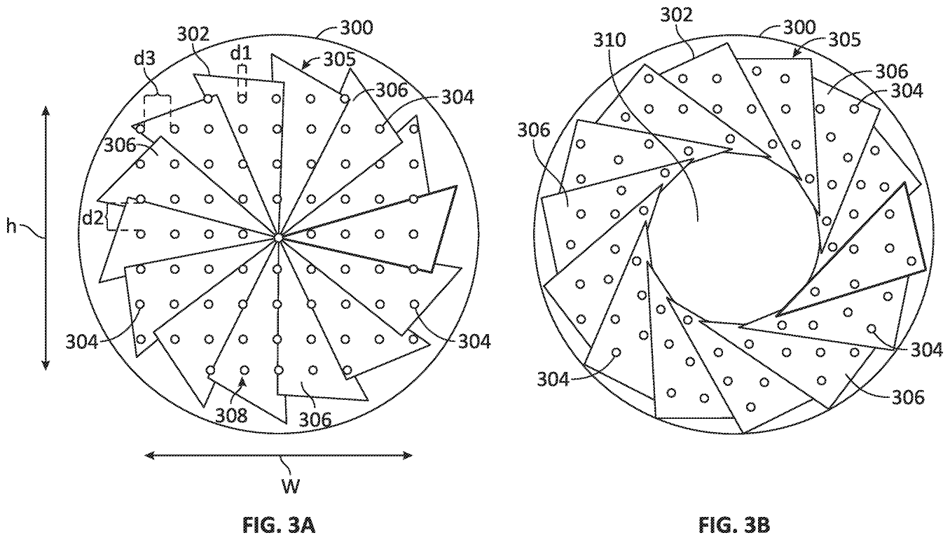

The pre-exposure device 108 is positioned within the path of the x-ray beam 114, between the object 112 to be imaged and the x-ray source 102, and is capable of partially blocking the x-ray beam 114 before it reaches the object 112. In embodiments, the pre-exposure device 108 may include a blocking component comprising a physical arrangement of blocking areas and transparent areas, wherein only the transparent areas allow passage of the x-ray beam 114 and all other areas block passage of the x-ray beam 114, for example, as shown in FIG. 2. The blocking component of the pre-exposure device 108 may be removable, or movable into and out of the path of the x-ray beam 114, so that the pre-exposure device 108 blocks the x-ray beam 114 only when needed. For example, the pre-exposure device 108 may be configured to have an active state that places the blocking component within the path of the x-ray beam 114, and an inactive state that removes the blocking component from the path of the x-ray beam 114. The pre-exposure device 108 may include an electromechanical or pneumatic mechanism that is configured to automatically move the blocking component out of the x-ray beam path in the inactive state, for example, as shown in FIGS. 3A and 3B.

In embodiments, the x-ray apparatus 100 can be configured to obtain or acquire a preliminary x-ray image while the pre-exposure device 108 is in the active state, or when the x-ray beam 114 is partially blocked. The preliminary image may be used by the x-ray apparatus 100 to determine certain information about the object 112 and/or the particular imaging set-up. The x-ray apparatus 100 can be further configured to use this information to improve or refine a main x-ray image for the object 112 that is acquired while the pre-exposure device 108 is in the inactive state, or when the x-ray beam 114 is unblocked. As a result, the x-ray apparatus 100 can be configured to perform two exposures for each object being imaged: a first or preliminary exposure (“pre-exposure”) and a second or main exposure. Both exposures may be “full” intensity exposures, and the two exposures may be performed back-to-back, or in quick succession (e.g., about one second apart). However, by activating the pre-exposure device 108 during the first exposure, the overall radiation load to the object 112 is actually reduced, for example, as compared to existing x-ray imaging systems that use an anti-scatter grid to remove scattered radiation and/or a dosimetry device for exposure correction.

For example, the pre-exposure information obtained during the first exposure may be used to calculate density information for the object 112 itself, thus taking into account the individual properties of the specific object 112 (or patient), such as, e.g., fat, muscle, and/or bone composition. This density information can be used to select appropriate exposure control parameters for the particular object 112, such as, for example, the exposure time (mS) and electrical quantities (kV, mA) applied to the x-ray tube 102. The corrected exposure control parameters can be applied during the second exposure, thus ensuring that during the main exposure, the object 112 is exposed to only the amount of radiation that is required for proper imaging of that particular object 112.

The pre-exposure information may also be used to remove, or minimize the impact of, scattered radiation in the main x-ray image, without requiring a separate anti-scatter grid. For example, the pre-exposure information can be used to estimate a scatter image for the object 112, or a representation of the scattered radiation that reaches the detector 110 when imaging the object 112, and the scatter image may be used to deduct scattered radiation from the x-ray image acquired during the main exposure. By eliminating the anti-scatter grid, the x-ray apparatus 100 can further reduce the radiation load to the patient. More specifically, in conventional x-ray systems, the radiation dose must be increased to account for the absorption of useful x-ray photons by the anti-scatter grid itself. This increase in radiation dose is not required for the x-ray apparatus 100 due to the absence of an anti-scatter grid. Moreover, the radiation load to the object 112 is reduced during pre-exposure due to the presence of the pre-exposure device 108, or more specifically, the blocking areas included therein.

In some cases, the pre-exposure information can also be used to determine whether the x-ray imaging detector 110 is properly positioned behind the object 112, or vice versa (i.e. whether the object 112 is properly positioned in front of the detector 110). If a misalignment is detected, the x-ray apparatus 100 can be configured to stop or prevent the main exposure, thus preventing an unnecessary radiation dose to the patient. More specifically, if the object 112 is not properly positioned on the detector 110 during main exposure, the resulting x-ray image may not be usable. In such cases, the main exposure would be repeated, after correcting the object-detector alignment, thus increasing the total radiation load to the patient or object 112. Accordingly, using the pre-exposure information gathered by the pre-exposure device 108 to check alignment before the main exposure can help reduce or minimize the total amount of radiation delivered to the patient.

FIGS. 1A, 1B, and 1C depict various alternative configurations for placing the pre-exposure device 108 between the patient 112 and the x-ray source 102 within the x-ray apparatus 100. In each case, the pre-exposure device 108 is disposed adjacent the collimator 104 or within the collimator 104 itself. For example, FIG. 1A illustrates an embodiment of the x-ray apparatus 100 in which the pre-exposure device 108 (also referred to herein as a “beam stopper”) is disposed at, or adjacent to, an exit or emitting end of the collimator 104, in order to intercept, or coincide with, the x-ray beam 114 as it exits the collimator 104. In such cases, the pre-exposure device 108 may be added to the x-ray apparatus 100 after manufacturing (e.g., retrofitted) and/or may be offered as an optional add-on or attachment for the x-ray apparatus 100 during purchase. Alternatively, the pre-exposure device 108 may be installed in the apparatus 100 during manufacturing. Regardless of when or how the device 108 is installed, the pre-exposure device 108 can be permanently attached or secured to the x-ray apparatus 100 at the time of imaging, to ensure proper alignment with the x-ray source 102.

FIG. 1B illustrates another embodiment of the x-ray apparatus 100 in which the pre-exposure device 108 is disposed between the collimator 104 and the x-ray source 102, for example, adjacent to an entrance end of the collimator 104 and/or adjacent to the output end 106 of the x-ray source 102, in order to intercept the x-ray beam 114 as it exits the x-ray source 102. FIG. 1C illustrates yet another embodiment of the x-ray apparatus 100 in which the pre-exposure device 108 is disposed within the collimator 104 itself, so as to intercept the x-ray beam 114 after it enters the collimator 104 and before it exits the same. In either of these embodiments, the pre-exposure device 108 may be a built-in component, or added to the x-ray apparatus 100 during manufacturing.

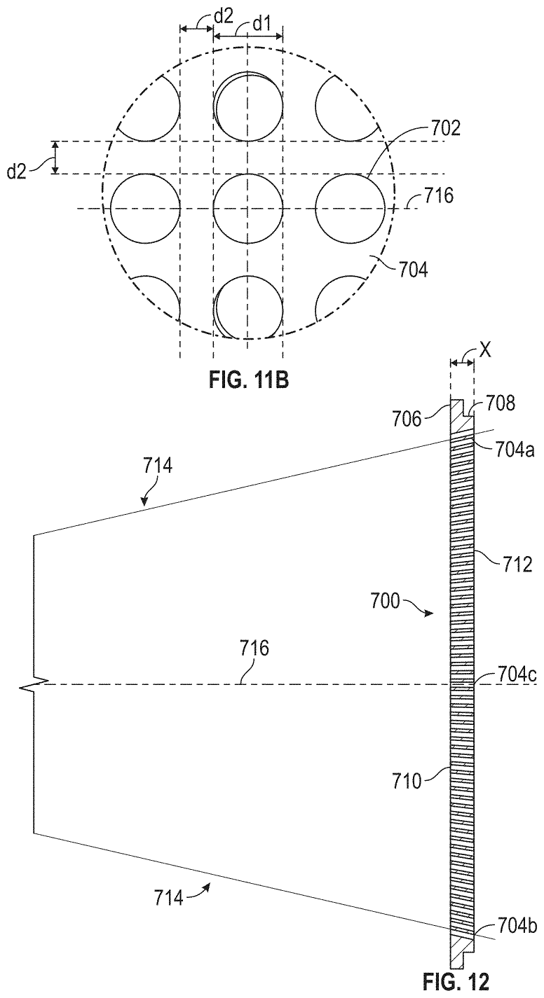

In embodiments, an exact location of the pre-exposure device 108 within the x-ray apparatus 100 may be selected based on the focal spot for the x-ray tube 102. For example, in FIG. 1A, the pre-exposure device 108 is placed adjacent the exit end of the collimator 104 to be as far as possible from the focal spot of the x-ray tube 102 in order to avoid penumbra, as described herein. In other embodiments, such as, e.g., FIG. 1B or 1C, penumbra may still be avoided at shorter distances from the focal spot by configuring the pre-exposure device 108 to be very precisely focused towards the x-ray tube focal spot, for example, as described herein with respect to FIGS. 11A, 11B, and 12.

FIG. 2 illustrates an exemplary blocking component 200 of a pre-exposure device configured to be positioned between an object to be imaged and an x-ray source (e.g., x-ray source 102 of FIG. 1A) in order to partially block an x-ray beam emitted by the x-ray source, in accordance with embodiments. For example, the blocking component 200 may be included in the pre-exposure device 108 of the x-ray apparatus 100, or other similar device, and may be positioned adjacent to an exit side of the collimator 104 (e.g., as shown in FIG. 1A), between the collimator 104 and the x-ray source 102 (e.g., as shown in FIG. 1B), or inside the collimator 104 (e.g., as shown in FIG. 1C).

In embodiments, an overall size, e.g., height, h, and width, w, of the blocking component 200 can be selected based on certain dimensions of the collimator, in order to fully capture the x-ray beam output by the x-ray source. Which dimensions play a factor can depend on where the blocking component 200 is located. For example, for the position shown in FIG. 1A, the blocking component 200 may be sized to completely cover, or at least coincide with a height and width of, the exit end of the collimator. As another example, for the position shown in FIG. 1B, the blocking component 200 may be sized to completely cover, or at least coincide with a height and width of, the entrance end of the collimator. As yet another example, for the position shown in FIG. 1C, the blocking component 200 may be sized to substantially coincide with a height and width of the collimator at the location of the blocking component 200, or at least fit inside the collimator.

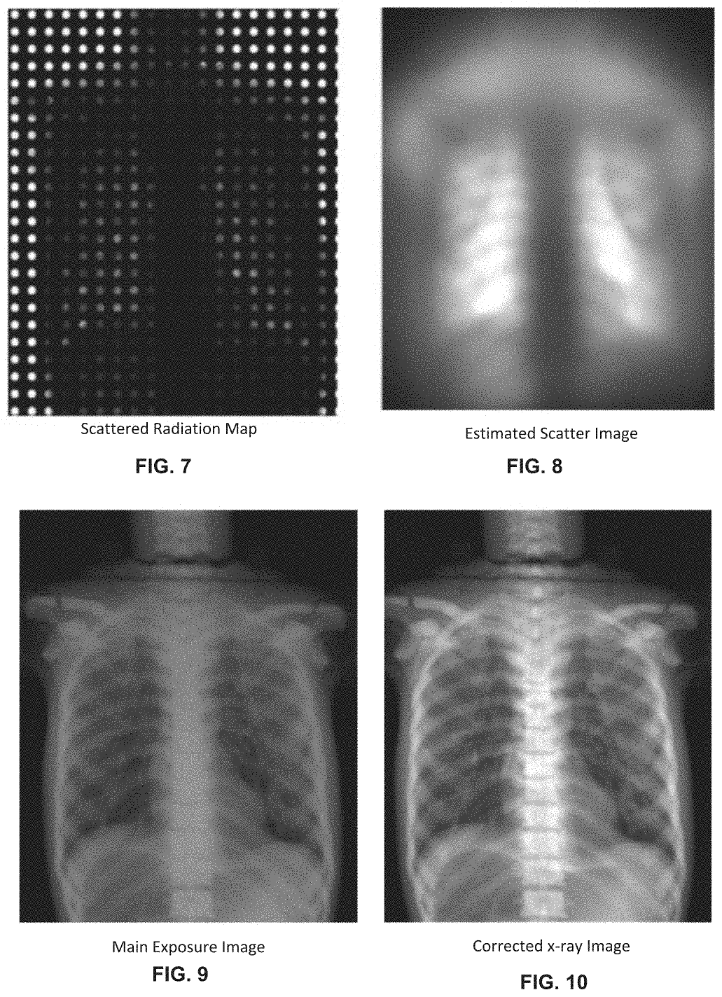

As shown in FIG. 2, the blocking component 200 comprises a solid material 202 configured to block passage of the x-ray beam and an arrangement of openings 204 disposed across an extent of the solid material 202 to allow passage of the x-ray beam through select areas. An x-ray imaging detector (e.g., detector 110 in FIG. 1A) picks up or captures the portions of the x-ray beam that pass through the transparent openings 204 and produces an image based thereon (e.g., the pre-exposure image). The detector image may show a high signal level for the areas corresponding to the openings 204 (e.g., due to detection of the x-ray beam) and a low or zero signal level for the areas corresponding to the solid material 202 (e.g., due to no detection of the x-ray beam). If there is no scattered radiation, the resulting detector image would look substantially similar to the pattern formed by the openings 204 in FIG. 2. In reality, the detector image includes one or more shadow zones, or areas in which scattered radiation from the x-ray beam is detected. The shadow zones may overlap with one or more high signal level areas, thus reducing the signal level for those areas and forming “shadowy” areas in the detector image, as shown in FIG. 7, for example.

According to embodiments, the solid material 202 may be comprised of lead, plastic, iron, tin, copper, aluminum, tungsten, or any other suitable material capable of completely absorbing x-radiation, or otherwise stopping an x-ray beam from passing through. In some cases, the solid material 202 may be a combination of two or more suitable materials. In one embodiment, the solid material 202 is at least partially made of tungsten and has a thickness or depth of about 2 millimeters (mm). In other cases, the solid material 202 of the blocking component 200 may be thicker or thinner (e.g., 1 mm or less).

While the illustrated embodiment shows the blocking component 200 as a sheet of blocking material 202 with perforations formed throughout the material 202 to create the openings 204, other configurations or structures for partially blocking the x-ray beam are also contemplated. For example, in some embodiments, the blocking component may be comprised of a plurality of thin, non-transparent rods made from the blocking material 202 and arranged vertically and horizontally to form a grid pattern. In such cases, the open spaces between the rods can serve as the openings 204 for allowing passage of the x-ray beam.

In embodiments, the openings (or apertures) 204 may be arranged in a pattern that is configured (e.g., sized and shaped) to span across, or coincide with, at least a characteristic extent of the x-ray beam, in order to ensure partial passage of the x-ray beam through the blocking component 200. For example, the number of openings 204 in the solid material 202 may be selected so that the pattern of openings 204 covers a field of view of the x-ray source, which is determined by a maximum size of the collimator attached to the x-ray source. As will be appreciated, the total number of openings 204 used to form this pattern may also depend on the size or diameter of each opening 204 and the separation distance or spacing between adjacent openings 204.

In the illustrated embodiment, the plurality of openings 204 are arranged in a uniform grid-like pattern with each opening 204 having a uniform diameter, d1, and a distance (or characteristic distance) between adjacent openings 204. In some embodiments, the pattern of openings 204 may be formed by selecting a vertical distance, d2, between adjacent openings 204 that is equal to a horizontal distance, d3, between adjacent openings 204, thus forming a substantially square or circular pattern, depending on the overall shape of the blocking component 200 and/or collimator opening. In other embodiments, the vertical separation distance, d2, may be different from the horizontal separation distance, d3, thus forming a substantially rectangular or oval pattern.

In embodiments, the diameter, d1, of each opening (or aperture) 204 and the separation distances, d2 and d3, between adjacent openings 204 can be selected to optimize the estimation of scattered radiation. For example, the diameter, d1, of the openings 204 may be selected to optimize a signal-to-noise ratio (“SNR”) of the shadow zone in the detector image. In particular, if the openings 204 are too small, the total amount of radiation that passes through the blocking component 200 will be so reduced that the amount of dispersed radiation will not be enough to estimate the scattered radiation. On the other hand, if the openings 204 are too large, the amount of radiation to the patient will be increased, and the shadow zone areas will be too large to be useful for estimating scattered radiation. In some embodiments, the opening diameter, d1, may be selected based on the focal spot of the x-ray tube (e.g., x-ray tube 102 in FIG. 1A). In particular, the diameter, d1, may be equal to or greater than, but not less than, a minimum focal spot size of the x-ray tube (e.g., the smallest size option) to ensure that the entire focal spot is able to pass through any given opening 204. As an example, the diameter, d1, may be any value between 0.5 mm and 5 mm, based on the known focal spot sizes for existing x-ray tubes, and in a preferred embodiment, the diameter, d1, may be any value from about 0.5 mm to about 2 mm.

Likewise, the separation distances between adjacent openings 204 may also be selected based on the focal spot of the x-ray tube. In particular, the distance between adjacent openings 204 may be equal to or greater than the minimum focal spot size of the x-ray tube to ensure that the entire focal spot is blocked by the blocking material 202 between any two adjacent openings 204. In addition, the separation distance may be configured to be equal to or greater than the aperture diameter, d1, in order to avoid penumbra, or a shadowing on the detector side that occurs when the openings 204 are too close together. In a preferred embodiment, the separation distances, d2 and d3, may be any value from about 0.5 mm to about 4 mm, depending on the value selected for the aperture diameter, d1.

It should be appreciated that FIG. 2 illustrates one exemplary embodiment for the blocking component of the pre-exposure device described herein and that other embodiments are also contemplated. For example, while the openings 204 shown in FIG. 2 have a generally circular shape, in other embodiments, the openings, or transparent areas, may have other, non-circular shapes, so long as the overall geometry of the transparent areas relative to the non-transparent areas is still configured to allow for estimation of the scattered radiation. As another example, while the blocking component 200 shown in FIG. 2 has a generally planar or flat shape, in other embodiments, the blocking component 200 may have other shapes, such as, e.g., a conical or curved shape, for example, due to a mechanical design of the pre-exposure device. Likewise, while the blocking component 200 has a substantially square shape, in other embodiments, the blocking component of the pre-exposure device may have other shapes, such as, e.g., circular, rectangular, triangular, and others.

FIGS. 3A and 3B illustrate another exemplary pre-exposure device 300 comprising a blocking component 302 configured to be selectively movable into and out of an x-ray beam path, in accordance with embodiments. More specifically, FIG. 3A shows the pre-exposure device 300 in an active state, wherein the blocking component 302 has been moved to a first position for placing the blocking component 302 within the path of the x-ray beam. FIG. 3B shows the pre-exposure device 300 in an inactive state, wherein the blocking component 302 has been moved to a second position for removing the blocking component 302 from the path of the x-ray beam. The pre-exposure device 300 may be configured for placement within an x-ray apparatus adjacent to or within a collimator of the apparatus, for example, similar to the pre-exposure device 108 shown in any one of FIG. 1A, 1B, or 1C.

As illustrated, the blocking component 302 comprises an arrangement of openings 304 configured to allow passage of the x-ray beam, like the openings 204 in FIG. 2, and a solid material configured to block passage of the x-ray beam, like the solid material 202 in FIG. 2. The openings 304 may be arranged across an extent of the blocking component 302 to form a particular pattern. A geometry of the openings 304 and the overall pattern formed by the openings 304 may be similar to that of the openings 204 and the grid or grid-like pattern created thereby in FIG. 2. For example, a diameter, d1, of each opening 304 may be equal to or greater than a minimum focal size of the x-ray tube (e.g., x-ray source 102 shown in FIG. 1A), and a spacing or separation distance between adjacent openings 304 (e.g., vertical distance, d2, and horizontal distance, d3) may be greater than the diameter of the openings 304, in order to optimize estimation of scattered radiation and avoid penumbra, as described herein. Moreover, an overall width, w, and height, h of the pattern may be selected to cover or allow partial passage of a characteristic extent of the x-ray beam through the blocking component 302.

In embodiments, the pre-exposure device 300 further comprises an electromechanical mechanism 305 configured to automatically move said blocking component 302 between the first position and the second position. For example, the pre-exposure device 300 may include an iris diaphragm, adjustable collimator, or other electromechanical mechanism 305 having a plurality of blades or moveable sections 306 adapted or configured to collectively form the blocking component 302. In such cases, the electromechanical mechanism 305 of the pre-exposure device 300 can place the blocking component 302 within the path of the x-ray beam by selectively closing the moveable sections 306, or moving the sections 306 towards each other until they form a substantially closed surface or wall 308, as shown in FIG. 3A. Likewise, the electromechanical mechanism 305 can remove the blocking component 302 from the pathway of the x-ray beam by selectively opening the moveable sections 306, or moving the sections 306 apart until a transparent gap or open cavity 310 is formed at a center of the blocking component 302, as shown in FIG. 3B. A size of the open cavity 310 may be configured (e.g., sized and shaped) so that the x-ray beam can pass through the cavity 310 without impinging on the surrounding moveable sections 306.

Continuing with the above example, each moveable section 306 may be made of the solid, non-transparent material (e.g., tungsten) required to block the x-ray beam (such as, e.g., solid material 202 in FIG. 2) and may be adapted or configured to include one or more of the openings 304 to allow passage of at least a portion of the x-ray beam there through when the electromechanical mechanism 305 is in a closed position. The one or more openings 304 may be disposed or arranged on each moveable section 306 such that, when the electromechanical mechanism 305 is in the closed position, the plurality of openings 304 move into alignment to collectively form the grid pattern of the blocking component 302 shown in FIG. 3A. This grid pattern may be removed from the x-ray beam path by moving the electromechanical mechanism 305 to an open position.

In embodiments, the electromechanical mechanism 305 can be configured to automatically move the moveable sections 306 between the closed position and the open position, as needed during imaging. For example, when moving from the open position to the closed position, each movable section 306 may be configured to rotate inwards or towards the center of the blocking component 302, until the grid pattern is formed or the cavity 310 is fully covered. When moving from the closed position to the open position, each moveable section 306 may be configured to rotate outwards or away from the center of the blocking component 302, and/or travel upwards or downwards, by a preset amount (e.g., distance and/or angle), until the open cavity 310 is formed at the center of the pre-exposure device 300. As shown in FIG. 3B, the moveable sections 306 may overlap or fold over each other in order to move to the open position. Though not shown, in one embodiment, the pre-exposure device 300 may include a solid cover or housing surrounding the cavity 310 to create a pocket or sleeve for receiving the moveable sections 306 when the blocking component 302 is in the open or second position.

While the embodiments described herein include a specific structure and/or mechanism for achieving the above, it is contemplated that other techniques or designs for selectively moving a partially transparent element into and out of the path of an x-ray beam may be used and are intended to be covered by the present disclosure. For example, though the illustrated embodiment shows an electromechanical mechanism with about a dozen moveable sections 306, other embodiments may have more or fewer moveable sections 306. In one example embodiment, the electromechanical mechanism may include two moveable sections that are configured to join together, e.g., at a mid-line, to form the wall 308 and slide apart to create the open cavity 310. In such cases, the two moveable sections can be situated vertically (e.g., like sliding doors), horizontally (e.g., like a shutter), or at an angle. In another example embodiment, the electromechanical mechanism may include a single moveable section configured (e.g., sized and shaped) to form the wall 308, by itself, when in the closed position. As another example, though the illustrated embodiment depicts moveable sections 306 with perforations or openings 304 formed into the solid material of each section 306, other embodiments may include moveable sections that are thin, solid rods, or bars. In such cases, the rods may be moved into a grid pattern by horizontally arranging a first group of rods, vertically arranging a second group of rods, and configuring or selecting the vertical and horizontal distances between adjacent rods to create the transparent openings 304 of the grid pattern.

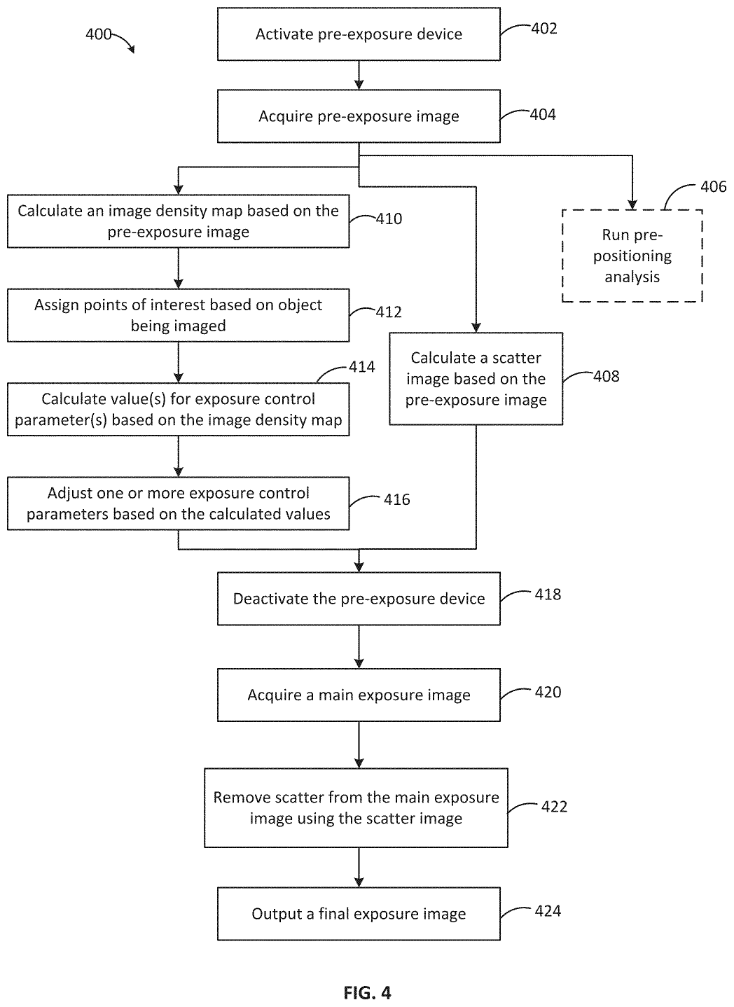

FIG. 4 illustrates an example method 400 of adjusting exposure in an x-ray imaging system, such as, for example, x-ray imaging system 600 shown in FIG. 6, or the x-ray apparatus 100 shown in FIG. 1, in accordance with embodiments. The method 400 can be implemented, at least in part, by at least one data processor executing software stored in memory, the processor and memory being included in the x-ray imaging system. More specifically, the method 400 can be carried out by at least one controller included in the x-ray imaging system, such as, for example, system controller 602, detector controller 604, and/or exposure controller 606 shown in FIG. 6, or the at least one controller included in the x-ray apparatus 100 of FIG. 1.

In order to carry out the operations of the method 400, the at least one controller may interact with one or more other components of the x-ray imaging system, such as, for example, an x-ray imaging detector (e.g., detector 110 of FIG. 1A), an x-ray source (e.g., x-ray tube 102 of FIG. 1A) configured to emit an x-ray beam (e.g., x-ray beam 114 of FIG. 1A) towards the x-ray imaging detector, a collimator (e.g., collimator 104 of FIG. 1A) configured to direct the x-ray beam towards the object to be imaged, and a pre-exposure device (e.g., pre-exposure device 108 of FIG. 1A) positioned adjacent to, or inside, the collimator and within a path of the x-ray beam. The pre-exposure device can include a blocking component (e.g., blocking component 200 of FIG. 2) configured to allow partial passage of the x-ray beam. For example, the blocking component may comprise a solid, non-transparent material (e.g., solid material 202 of FIG. 2) and an arrangement of transparent openings (e.g., openings 204 of FIG. 2) across an extent of the solid material. The pre-exposure device may further include an electromechanical mechanism (e.g., mechanism 305 of FIGS. 3A and 3B) configured to selectively move the blocking component into and out of the path of the x-ray beam.

The method 400 can begin at step 402, where the at least one controller activates the pre-exposure device. For example, the at least one controller (e.g., system controller 602) may activate the pre-exposure device by causing or instructing the blocking component to move into the path of the x-ray beam, so that the x-ray beam is partially blocked by the blocking component.

The method 400 further includes, at step 404, acquiring a pre-exposure image, using the x-ray imaging detector, while the pre-exposure device is active. In embodiments, acquiring the pre-exposure image includes using the at least one controller (e.g., exposure controller 606) to cause or instruct the x-ray source to emit an x-ray beam, or perform a preliminary exposure, while the pre-exposure device is partially blocking the beam, and using the at least one controller (e.g., detector controller 604) to cause or instruct the x-ray imaging detector to obtain or capture an x-ray image of an object (e.g., object 112 of FIG. 1A) disposed within the path of the pre-exposure beam.

As shown in FIG. 4, from step 404, the method 400 continues along at least one of three possible paths, namely step 406, step 408, and/or steps 410-416, to process the pre-exposure image and/or information obtained at step 404. In a preferred embodiment, the method 400 carries out all three paths or actions in parallel, either simultaneously or nearly simultaneously. For example, all the three actions may be completed within about one second or less, such that the operator barely notices a gap between the preliminary exposure at step 404 and a main exposure at step 420.

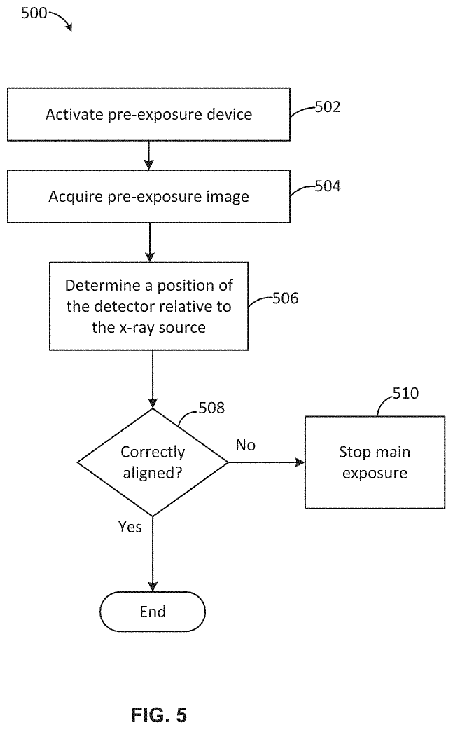

At step 406, the at least one controller runs a pre-positioning analysis, such as, e.g., method 500 of FIG. 5, for determining a position of the x-ray image detector relative to the x-ray source and/or the object to be imaged, and if the detector is misaligned, preventing acquisition of the main exposure image at step 420. The below description of FIG. 5 provides a more detailed discussion of the pre-positioning analysis.

At step 408, the at least one controller calculates a scatter image based on the pre-exposure image of the object to be imaged. In particular, the at least one controller (e.g., the detector controller 604) may be configured to use the information acquired during pre-exposure by the x-ray imaging detector to measure scattered radiation (or scatter) from the object to be imaged and generate a scatter map representing the distribution of scattered radiation. In embodiments, the scatter information may be collected against, or relative to, the shadow areas of the pre-exposure image, which correspond to the non-transparent areas of the blocking component. This is because, while the x-ray beam is expected to pass through the openings in the blocking component, only scattered photons will reach the shadow zone of the x-ray image detector, or the areas corresponding to the solid material.

As an example, FIG. 7 illustrates an exemplary scattered radiation map (or “scatter map”) generated from pre-exposure of the chest area of a patient using a blocking component having a grid pattern similar to the pattern shown in FIG. 2. In FIG. 7, the bright areas represent the x-ray beam passing through the openings of the blocking component unperturbed, the dark areas represent the x-ray beam being blocked or absorbed completely by the blocking component, and the gray or shadowy areas may represent areas where scatter photons reached the detector.

Referring back to FIG. 4, in embodiments, the scatter map may represent measured scatter from the patient in a limited or small number of points. For example, a previously-generated shadow zone mask may be used to provide a threshold or base comparison point for any given scatter map. The shadow zone mask may be generated during set-up or calibration by exposing the blocking component without a patient or object disposed between the pre-exposure device and the detector. During the pre-exposure stage, the shadow zone mask may be compared to the pre-exposure image in order to identify and select samples from the pre-exposure image that necessarily correspond to the scattered radiation map.

Due to the low frequency nature of the measured scatter, step 408 may also include interpolating information between the measured points of the scatter map to restore or estimate a complete scatter image. For example, FIG. 8 depicts an exemplary scatter image estimated based on the scatter map of FIG. 7. The scatter image may be used to correct, or adjust a contrast of, the main exposure image obtained at step 420, as described with respect to step 422.

At steps 410-416, the at least one controller (e.g., system controller 602) performs an image density analysis based on the pre-exposure image or information obtained at step 404. The results of this analysis can be used to adjust one or more exposure control parameters of the x-ray source, prior to acquiring a main exposure image at step 420, as described below.

More specifically, at step 410, the at least one controller (e.g., system controller 602) calculates an image density map based on the pre-exposure image obtained at step 404. The image density map may be generated based on information in the pre-exposure image that corresponds to the matrix of openings in the blocking component and thus, represents the density of the patient or object to be imaged. For example, because the openings are configured to allows passage of the x-ray beam, the signal values detected by the x-ray imaging detector in the areas corresponding to the openings may be indicative or representative of the density of the corresponding area of the patient.

At step 412, the at least one controller assigns one or more points or regions of interest to the image density map based on characteristics of, or an identification of, the object to be imaged. More specifically, the pre-exposure image may contain information regarding the overall shape of the object within the x-ray system's field of view. This information may be enough to identify which organ or body part is within the field of view. For example, one or more standard positioning templates for select body parts may be stored in a memory of the x-ray system and compared to the pre-exposure image to identify the organ, body part, or region in the field of view of the x-ray system. The selected template may be superimposed with the pre-exposure image to identify the one or more points on the image density map that represent or correspond to the object being imaged. These points (or points of interest) on the density map are then analyzed to determine the signal level at each point.

At step 414, the at least one controller calculates or determines one or more values for one or more exposure control parameters based on the image density map, or more specifically, the signal levels determined at step 412 for the relevant points of interest. More specifically, the at least one controller can calculate values for select exposure control parameters (or “exposure factors”) that will result in optimal values for the signal levels of the object to be imaged, particularly for the x-ray imaging detector being used. The optimal signal level value for a given point of interest may vary depending on the area of the body or body part and the expected density of that area (e.g., based on the standard template). For example, the density of the lungs is normally lower than that of surrounding tissues, as will be appreciated.

At step 416, the at least one controller (e.g., the exposure controller 606) adjusts the one or more exposure control parameters based on the values calculated at step 414. In embodiments, the exposure control parameters may comprise one or more electrical quantities applied to the x-ray tube, such as, e.g., anode current level (mA), electric potential (kV), and exposure time (mS) of the x-ray tube. In one exemplary embodiment, the exposure control parameters adjusted at step 416 comprise exposure time and anode current level. Thus, at step 416, the at least one controller can automatically control or adjust the amount of radiation that is optimal for imaging the given object using the x-ray imaging detector included in the x-ray system.

At step 418, the at least one controller deactivates the pre-exposure device. For example, the at least one controller (e.g., system controller 602) may cause or instruct the blocking component to move out of the path of the x-ray beam, so that the x-ray beam is no longer blocked by the blocking component. In some embodiments, step 418 may occur at the same time as, or nearly simultaneously with, one or more of the above pre-exposure processing paths, namely step 406, step 408, and steps 410-416. For example, the pre-exposure device may be deactivated while the at least one controller analyzes the pre-exposure image and/or information obtained during the pre-exposure at step 404. In other embodiments, step 418 may occur before such analysis occurs, or after the analysis is complete, as shown in FIG. 4.

At step 420, the method 400 includes acquiring a main exposure image, using the x-ray imaging detector, while the pre-exposure device is inactive. In embodiments, acquiring the main exposure image includes using the at least one controller (e.g., exposure controller 606) to cause or instruct the x-ray source to emit a second x-ray beam, or perform a main exposure, and using the at least one controller (e.g., detector controller 604) to cause or instruct the x-ray imaging detector to obtain or capture a second x-ray image of the object (e.g., object 112 of FIG. 1A) disposed within the path of the main x-ray beam. FIG. 9 illustrates an exemplary main exposure image of the chest area of a patient.

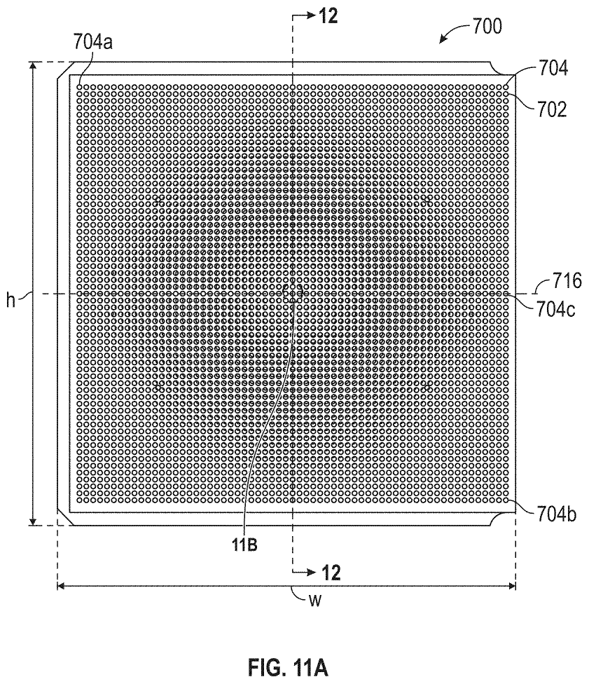

At step 422, the at least one controller (e.g., system controller) uses the scatter image to remove scatter from the main exposure image. For example, the at least one controller may subtract or deduct the scatter image generated at step 408 from the main exposure image generated at step 420 to obtain a corrected x-ray image. FIG. 10 illustrates an exemplary corrected x-ray image generated by subtracting the scatter image of FIG. 8 from the main exposure image of FIG. 9. In embodiments, removing scatter from the main exposure image can significantly improve a contrast level of the final x-ray image, as evidenced by comparing FIGS. 9 and 10, for example.

At step 424, the at least one controller (e.g., system controller) outputs the corrected x-ray image as a final exposure image for the object undergoing diagnostic imaging. For example, the at least one controller may output or provide the final exposure image to an image processor (e.g., processor 608) and/or display screen (e.g., display screen 614) of the x-ray system. The method 400 may end after step 424.

Referring now to FIG. 5, shown is an exemplary method of analyzing a position of an x-ray detector in an x-ray imaging system, relative to an x-ray emitting portion or source of the system, in accordance with certain embodiments. The method 500 may be implemented, at least in part, by at least one data processor executing software stored in a memory, the processor and memory being included in the x-ray imaging system. More specifically, the method 500 can be carried out by at least one controller included in the x-ray imaging system (e.g., x-ray system 600 of FIG. 6) and/or an x-ray apparatus (e.g., x-ray apparatus 100 of FIG. 1A). In some embodiments, the method 500 is performed as part of the method 400, for example, at step 406, as shown in FIG. 4. In other embodiments, the method 500 can be performed independently of, or simultaneously with, the method 400.

As shown, the method 500 includes, at step 502, activating a pre-exposure device, such as, e.g., pre-exposure device 108 of FIG. 1A, 1B, or 1C, similar to step 402 of method 400. At step 504, the at least one controller acquires a pre-exposure image by exposing the object (e.g., object 112 of FIG. 1A) to an x-ray beam from an x-ray source (e.g., x-ray tube 102 of FIG. 1A) while the pre-exposure device is activated, similar to step 404 of method 400. At step 506, the at least one controller uses the pre-exposure image to determine or identify a position of an x-ray imaging detector (e.g., detector 110 of FIG. 1A) behind the object to be imaged relative to the x-ray source. And at step 508, the at least one controller determines whether the identified detector position is correctly aligned or misaligned with the x-ray source position.

In embodiments, the detector position may be correct when the detector is positioned exactly perpendicular to (or at 90 degrees relative to) the x-ray source and/or the x-ray beam emitted thereby. The pre-exposure image may contain information that can identify whether or not the detector is positioned at 90 degrees. For example, a distance between the areas representing the openings in the blocking component may be uniform when the detector is perpendicular to the source and may be non-uniform when the detector is tilted or off-axis. In a preferred embodiment, the at least one controller can be configured (e.g., using software) to analyze the hole or opening distribution depicted in the pre-exposure image and measure or calculate spatial separation distances between the various openings, at step 506. Then at step 508, the at least one controller can be further configured to determine, based on the measured spatial separation distances, whether or not the detector is tilted or properly aligned. This may include, for example, comparing the measured distances to a preset or expected spatial separation distance and determining that the detector is misaligned if the measured distance does not match the known distance. Likewise, the at least one controller may determine that the detector is properly aligned if the measured value matches, or substantially matches, the known value.

If the detector is misaligned (or “No” at step 508), the method 500 continues to step 510, where the at least one controller prevents the x-ray apparatus, or x-ray source, from performing the main exposure. If the detector is correctly aligned (or “Yes” at step 508), the method 500 may end after step 508 or, in cases where the method 500 is performed as a subset of the method 400, may continue from step 508 to step 418 of method 400.

FIG. 6 is a block diagram of an exemplary x-ray system 600 configured to carry out one or more techniques for controlling an x-ray apparatus, such as, e.g., method 400 of FIG. 4 and/or method 500 of FIG. 5, in accordance with embodiments. In some cases, the x-ray apparatus may be included in the x-ray system 600, so as to form a single x-ray device. In other cases, the x-ray system 600 may be separate from the x-ray apparatus, but still communicatively coupled thereto. In such cases, the x-ray system 600 may transmit instructions to the x-ray apparatus, for example, in order to control exposure parameters and/or enable access to one or more components of the x-ray apparatus, in accordance with the techniques described herein. In still other cases, select portions of the x-ray system 600 may be incorporated into the x-ray apparatus, while remaining portions of the x-ray system 600 may be disposed separately from the x-ray apparatus, for example, in a control unit (e.g., operating console or computer) communicatively coupled to the x-ray apparatus.

In the illustrated embodiment, the x-ray system 600 is shown as being communicatively coupled to the x-ray apparatus 100 of FIG. 1A, or more specifically, select components thereof, such as the x-ray imaging detector 110, the x-ray source 102, and the pre-exposure device 108. In other embodiments, the x-ray system 600 may comprise, or may be communicatively coupled to, a different x-ray apparatus that includes an pre-exposure device positioned between the object to be imaged and the output end of the x-ray source and comprising a blocking component configured to partially block the x-ray beam during a first exposure and permit complete passage of the x-ray beam during a second or main exposure.

In some embodiments, the x-ray system 600 may be representative of a computer utilized system to implement method 400 shown in FIG. 4 and/or method 500 of FIG. 5. The x-ray system 600 can include any type of computing device, including one or more special or general purpose digital computer(s), such as a mainframe computer, a personal computer (desktop, laptop, tablet-type, or otherwise), a workstation, a minicomputer, a computer network, a “virtual network,” a “internet cloud computing facility,” a personal digital assistant, a smartphone, a tablet, or other handheld or mobile computing device.

According to embodiments, the x-ray system 600 includes one or more processors 608, and a memory device 610, user interface 612, and display device 614 coupled to the one or more processors 608. The user interface 612 can include one or more input devices (e.g., a keyboard, a mouse, a touch screen, a microphone, a stylus, a radio-frequency device reader, and the like) for receiving inputs from the user or other sources. In embodiments, the user interface 612 includes an exposure switch (or button or other input device) for controlling operation of the x-ray source 102, such as, e.g., initiating a first or preliminary exposure and initiating a second or main exposure. The display device 614 can include any type of display screen for displaying content to the user, such as the x-ray images obtained by the detector 110.

Though not shown, the x-ray system 600 may also include an input and/or output (I/O) portion communicatively coupled to the processor(s) 608 and to the user interface 612 and display device 614, so that a command or other input entered or provided by a user through the user interface 612 can be forwarded to the processor 608 via the I/O portion, for example, and/or an output generated by the processor 608 can be provided to the display device 614 for display via the I/O portion. In some embodiments, the x-ray system 600 can include a communications module (not shown) comprising one or more transceivers and/or other devices for communicating with one or more networks (e.g., a wide area network (including the Internet), a local area network, a GPS network, a cellular network, a Bluetooth network, other personal area network, and the like).

The one or more processors 608 can be hardware devices for executing software, particularly software stored in memory device 610, some of which may or may not be unique to the x-ray apparatus 100 shown in FIG. 1. Each processor 608 can be any custom-made or commercially available processor, a central processing unit (CPU), an auxiliary processor among several processors associated with the x-ray system 600, a semiconductor-based microprocessor (in the form of a microchip or chip set), another type of microprocessor, or generally any device for executing software instructions. The processor(s) 608 may also represent a distributed processing architecture such as, but not limited to, SQL, Smalltalk, APL, KLisp, Snobol, Developer 200, MUMPS/Magic. In some embodiments, the one or more processors 608 includes at least one image processor for collecting, processing, and enhancing an x-ray image signal or information received from the imaging detector 110. The processed image may be displayed on the display device 614 and stored in memory device 610.

Memory device 610 can include any one or a combination of volatile memory elements (e.g., random access memory (RAM, such as DRAM, SRAM, SDRAM, etc.)) and nonvolatile memory elements (e.g., ROM, hard drive, tape, CDROM, etc.). Moreover, the memory 610 may incorporate electronic, magnetic, optical, and/or other types of storage media. The memory device 610 can have a distributed architecture where various components are situated remote from one another, but are still accessed by the processor 608. The memory 610 may store software that includes one or more separate programs comprising ordered listings of executable instructions for implementing logical functions.

When the x-ray system 600 is in operation, the one or more processors 608 can be configured to execute software stored within the memory device 610, to communicate data to and from the memory 610, and to generally control operations of the x-ray system 600 pursuant to the software. In some embodiments, the memory 610 includes a non-transitory computer readable medium for implementing all or a portion of method 400 shown in FIG. 4 and/or method 500 shown in FIG. 5. The memory portion 610 may also be used to implement at least part of one or more databases utilized by the x-ray system 600, such as, for example, an x-ray imaging database for storing x-ray images and/or information related thereto. In addition, the memory 610 can store one or more executable computer programs or software modules comprising a set of instructions to be performed, such as, for example, a pre-exposure control application that may be executed by the computer processor 608 to carry out the principles disclosed herein (e.g., methods 400 and/or 500). The executable programs can be implemented in software, firmware, hardware, or a combination thereof.

The x-ray imaging system 600 further comprises one or more controllers or control modules comprising circuitry or electronics configured to control specific components of the x-ray apparatus 100. For example, as shown in FIG. 6, the x-ray imaging system 600 can include system controller 602, which is communicatively coupled to the one or more processors 608 and the user interface 612. System controller 602 can be configured to govern the overall operation of the x-ray apparatus 100, for example, based on instructions received from the processor(s) 608 and/or commands received from the user via the user interface 612 (e.g., start exposure, stop exposure, etc.).

The x-ray imaging system 600 can also include detector controller 604, which is communicatively coupled to the system controller 602, x-ray imaging detector 110, and processor(s) 608. The detector controller 604 can be configured to control operation of the x-ray imaging detector 110 to read out a signal from each element of the detector 110 exposed to the x-ray beam 114 and acquire an image based thereon, in accordance with instructions received from the system controller 602. The detector controller 604 may also provide x-ray images and other information to the one or more processor(s) 608, such as, e.g., an image processor included therein for processing the x-ray imaging signal provided by the detector 110.

The x-ray system 600 can further comprise exposure controller 606, which is communicatively coupled to the system controller 602 and the x-ray source 102. The exposure controller 606 can be configured to control operation of the x-ray source 102 to generate an appropriate x-ray exposure dosage based on instructions received from the system controller 602, such as, e.g., when to start or stop an exposure, what values to apply for the exposure control parameters of the x-ray source (e.g., kV, mA, and mS), etc. Though FIG. 6 shows several separate controllers 602, 604, and 606, in other embodiments, two or more of the controllers may be consolidated into one control device, such as, e.g., a system and detector controller, a system and exposure controller, etc.

FIGS. 11A, 11B, and 12 illustrate another exemplary blocking component 700 of a pre-exposure device configured to be positioned between an object to be imaged and an x-ray source in order to partially block an x-ray beam emitted by the x-ray source, in accordance with embodiments. An overall operation of the blocking component 700 may be substantially similar to the overall operation of the blocking component 200 described herein. For example, like the blocking component 200, the blocking component 700 may be included in the pre-exposure device 108 of the x-ray apparatus 100 shown in FIG. 1, or other similar device, and may be positioned adjacent to an exit side of the collimator 104 (e.g., as shown in FIG. 1A), between the collimator 104 and the x-ray source 102 (e.g., as shown in FIG. 1B), or inside the collimator 104 (e.g., as shown in FIG. 1C).

Also like the blocking component 200, an overall size and shape of the blocking component 700 can be selected based on certain dimensions of the collimator 104 (i.e. depending on its location relative to the collimator 104), so that the blocking component 700 fully captures the x-ray beam being output by the x-ray source. For example, in some embodiments, the blocking component 700 is generally square-shaped to match a generally square-shaped body, or cross-section, of the collimator 104. For example, in one embodiment, the blocking component 700 has an overall height, h, of about 53 to 54 mm and an overall width, w, of about 53 to 54 mm. The blocking component 700 also has a depth or thickness, x, that may be selected based certain dimensions of the pre-exposure device 108 and/or the collimator 104, or the mechanism for coupling the blocking component 700 thereto. For example, in some embodiments, the blocking component 700 has a thickness, x, of about 2 to 3 mm.

In a preferred embodiment, the blocking component 700 is configured for placement inside the collimator 104. In such cases, the collimator 104 may include a slot, groove, channel, or other receiving area (not shown) configured to hold or place the blocking component 700 within the pathway of the x-ray beam, when the pre-exposure device 108 is in the active state. And the blocking component 700 may include a tab, lip, or other projection 706 configured to engage the receiving area of the collimator 104 or otherwise keep the blocking component 700 in place during use. For example, in the illustrated embodiment, the tab 706 extends out from a main body 708 of the blocking component 700, so as to form a border having a characteristic height around the main body 708. In such cases, the receiving area of the collimator 104 may include a channel or groove having a height selected based on said characteristic height and a depth selected based on a depth or thickness of the tab 706, or may be otherwise configured to receive said tab 706. In one embodiment, the tab 706 has a thickness of about 1 mm, while the thickness, x, of the overall blocking component 700 is about 2.2 mm. In embodiments, the blocking component 700 may be removed from said receiving area of the collimator 104 when the pre-exposure device 108 is in the inactive state, for example, using a sliding mechanism or other suitable mechanism configured to automatically move (e.g., push or pull) the blocking component 700 out of the x-ray beam path in response to a control signal (e.g., from the system controller 602).

Also like the blocking component 200, the blocking component 700 comprises a solid material 702 (or blocking material) configured to block passage of the x-ray beam and an arrangement of a plurality of openings 704 (or transparent areas) disposed across an extent of the solid material 702 to allow passage of the x-ray beam through select areas. The portions of the x-ray beam that pass through the transparent openings 704 are picked up or captured by an x-ray imaging detector (e.g., such as detector 110) and used to produce an image (e.g., the pre-exposure image described herein). As with blocking component 200, the resulting detector image will show a low or zero signal level for the areas corresponding to the solid material 702, due to an absence of the x-ray beam, and a high signal level for the areas corresponding to the openings 704, due to detection of the x-ray beam. The detector image will also include one or more shadow zones in the areas where scattered radiation from the x-ray beam is detected.

According to embodiments, the solid material 702 may be comprised of lead, plastic, iron, tin, copper, aluminum, tungsten, or any other suitable material capable of completely absorbing x-radiation, or otherwise stopping an x-ray beam from passing through. In some cases, the solid material 702 may be a combination of two or more suitable materials. In one embodiment, the solid material 702 is a high-detail stainless steel made from a combination of boron, silicon, aluminum, chromium, nickel, and molybdenum, with a thickness or depth of about 2 to 2.5 millimeters (mm).

As shown in FIG. 11B, each of the openings (or apertures) 704 can be substantially circular with a uniform diameter, d1, and can be disposed equidistant from each other, or separated by a uniform distance, d2, both vertically and horizontally, so as to be arranged in a uniform grid pattern across a substantial extent of the blocking component 700. The exact values for d1 and d2 may be selected to avoid penumbra and optimize the estimation of scattered radiation, as described herein with respect to the blocking component 200. For example, in a preferred embodiment, the diameter, d1, of the openings 704 is about 0.5 mm, based on a minimum focal spot size of the x-ray tube, and the distance, d2, between adjacent openings 704 is about 0.3 mm, so that the center to center distance between adjacent openings 704 is greater than the minimum focal spot size. In embodiments, the blocking component 700 may be manufactured with a very high level of precision in order to comply with the very low tolerances for the positioning of each opening 704 and the distance, d2, between them.

As shown in FIG. 12, the openings 704 extend from a first face 710 of the blocking component 700 to an opposing, second face 712 of the blocking component 700 (or across the entire thickness, x, of the blocking component 700), such that both of the faces 710 and 712 are perforated. This enables an x-ray beam 714 emitted by the x-ray source (e.g., similar to x-ray beam 114 shown in FIG. 1) to pass all the way through the blocking component 700, or at least the transparent areas thereof. In embodiments, the blocking component 700 is configured to guide the x-ray beam 714 as it passes through the openings 704 to further avoid penumbra, or shadowing at the detector side. In particular, looking from the second face 712 to the first face 710, the openings 704 can be angled or focused towards the x-ray tube focal spot, so that each opening 704 substantially matches, or coincides with, an angle of the x-ray beam 714 as it passes through that particular opening 704. In this manner, the x-ray beam 714 can pass through the transparent areas of the blocking component 700 substantially unperturbed, and scattered radiation is unable, or less likely, to pass through the blocking component 700 and be detected by the detector.

As shown in FIG. 12, this focused orientation is achieved by extending the openings 704 at various angles relative to a central axis 716 of the blocking component 700. The exact angle selected for each opening 704 can depend on the location of the opening 704 within the blocking component 700 and a distance from the blocking component 700 to the focal spot of the x-ray tube. As an example, openings 704a along a top row of the blocking component 700 may extend at a first angle relative to the central axis 716, while openings 704b along a bottom row of the blocking component 700 may extend at a second angle relative to the central axis 716 that is equal in magnitude but opposite in direction as the first angle, wherein the first and second angles represent the outskirts or outer edges of the x-ray beam 714. Conversely, openings 704c along a central row (or rows) of the blocking component 700 may extend at a zero degree angle relative to the central axis 716, since the x-ray beam 714 is directed perpendicular to a center of the blocking component 700. In one embodiment, the openings 704 are configured for a distance of about 97 mm between the blocking component 700 and the x-ray tube focal spot.

In certain embodiments, the process descriptions or blocks in the figures, such as FIG. 4 and FIG. 5, can represent modules, segments, or portions of code which include one or more executable instructions for implementing specific logical functions or steps in the process. Any alternate implementations are included within the scope of the embodiments described herein, in which functions may be executed out of order from that shown or discussed, including substantially concurrently or in reverse order, depending on the functionality involved, as would be understood by those having ordinary skill in the art.

It should be emphasized that the above-described embodiments, particularly, any “preferred” embodiments, are possible examples of implementations, merely set forth for a clear understanding of the principles of the invention. Many variations and modifications may be made to the above-described embodiment(s) without substantially departing from the spirit and principles of the techniques described herein. All such modifications are intended to be included herein within the scope of this disclosure and protected by the following claims.

1. An x-ray imaging system, comprising:
an x-ray source configured to emit an x-ray beam towards an x-ray imaging detector;
the x-ray imaging detector configured to obtain an x-ray image of an object placed adjacent to the x-ray imaging detector and at least partially within a path of the x-ray beam; and
a pre-exposure device positioned between said object and the x-ray source and comprising a blocking component including an arrangement of openings across an extent of the component, the pre-exposure device having an active state wherein said blocking component is configured to reside within the path of the x-ray beam and an inactive state wherein said blocking component is configured to not reside in the path of the x-ray beam.

2. The x-ray imaging system of claim 1, wherein said openings are configured to allow passage of the x-ray beam when the pre-exposure device is in the active state.

3. The x-ray imaging system of claim 2, wherein said blocking component further includes a solid material configured to block passage of the x-ray beam when the pre-exposure device is in the active state, said arrangement of openings being disposed in the solid material.

4. The x-ray imaging system of claim 1, wherein the pre-exposure device further comprises a mechanism configured to automatically move the blocking component out of the path of the x-ray beam in the inactive state.

5. The x-ray imaging system of claim 1, wherein the x-ray imaging detector is configured to acquire a pre-exposure image while the pre-exposure device is in the active state and acquire a main exposure image while the pre-exposure device is in the inactive state.

6. The x-ray imaging system of claim 5, further comprising at least one controller configured to control operation of the pre-exposure device and process the images acquired by the detector.

7. The x-ray imaging system of claim 6, wherein the at least one controller is further configured to adjust one or more exposure control parameters based on the pre-exposure image and prior to acquiring the main exposure image.

8. The x-ray imaging system of claim 7, wherein the at least one controller is further configured to calculate an image density map based on the pre-exposure image and determine one or more values for the one or more exposure control parameters based on the image density map.

9. The x-ray imaging system of claim 7, wherein the exposure control parameters comprise one or more of exposure time and anode current level.

10. The x-ray imaging system of claim 6, wherein the at least one controller is further configured to calculate a scatter image based on the pre-exposure image, and generate a corrected x-ray image by using the scatter image to remove scatter from the main exposure image.

11. The x-ray imaging system of claim 6, wherein the at least one controller is further configured to determine a position of the detector relative to the x-ray source, and if the detector is misaligned, prevent acquisition of the main exposure image.

12. An x-ray apparatus, comprising:
an x-ray source configured to emit an x-ray beam;
an x-ray imaging detector configured to obtain an x-ray image of an object placed adjacent to the x-ray imaging detector and at least partially within a path of the x-ray beam;
a collimator disposed adjacent to an output end of the x-ray source; and
a pre-exposure device positioned between said object and the x-ray source and comprising a blocking component including an arrangement of a plurality of openings across an extent of the component, said blocking component being selectively movable to a first position for placing the component within the path of the x-ray beam and to a second position for removing the component from the path of the x-ray beam.

13. The x-ray apparatus of claim 12, wherein the pre-exposure device further comprises an electromechanical mechanism configured to move said blocking component between the first position and the second position.

14. The x-ray apparatus of claim 12, wherein said plurality of openings are configured to allow passage of the x-ray beam when the blocking component is in the first position.

15. The x-ray apparatus of claim 12, wherein said blocking component further includes a solid material configured to block passage of the x-ray beam when in the first position, said arrangement of openings being disposed in the solid material.

16. The x-ray apparatus of claim 12, wherein said plurality of openings are angled towards a focal point of the x-ray source.

17. The x-ray apparatus of claim 12, wherein the pre-exposure device is disposed adjacent to the collimator.

18. The x-ray apparatus of claim 12, wherein the pre-exposure device is disposed within the collimator.

19. A method of adjusting exposure in an x-ray imaging system comprising at least one controller, an x-ray imaging detector, an x-ray source configured to emit an x-ray beam towards the x-ray imaging detector, and a pre-exposure device positioned adjacent the x-ray source and within a path of the x-ray beam, the pre-exposure device including a blocking component having an arrangement of openings across an extent of the component to partially block the x-ray beam, the method comprising:
activating the pre-exposure device using the at least one controller;
acquiring a pre-exposure image, using the x-ray imaging detector, while the pre-exposure device is active;
deactivating the pre-exposure device using the at least one controller; and
acquiring a main exposure image, using the x-ray imaging detector, while the pre-exposure device is inactive.

20. The method of claim 19, further comprising: prior to acquiring the main exposure image, adjusting one or more exposure control parameters, using the at least one controller, based on the pre-exposure image.

21. The method of claim 20, wherein adjusting the one or more exposure control parameters includes calculating an image density map based on the pre-exposure image, and determining one or more values for the one or more exposure control parameters based on the image density map.

22. The method of claim 20, wherein the exposure control parameters comprise one or more of exposure time and anode current level.

23. The method of claim 19, further comprising: calculating, using the at least one controller, a scatter image based on the pre-exposure image, and generating, using the at least one controller, a corrected x-ray image by using the scatter image to remove scatter from the main exposure image.

24. The method of claim 19, further comprising: determining, using the at least one controller, a position of the detector relative to the x-ray source, and if the detector is misaligned, preventing acquisition of the main exposure image.

25. The method of claim 19, wherein activating the pre-exposure device includes causing said blocking component to move into the path of the x-ray beam, and deactivating the pre-exposure device includes causing said blocking component to move out of the path of the x-ray beam.
